(19) United States
(12) Patent Application Publication
(10) Pub. No.: US 2026/0355442 A1
(43) Pub. Date: Nov. 12, 2026

(54) Low-Noise Sound-muffling Box for Ventilator, and Ventilator

(71) Applicant: WEIHAI WEIGAO HEALTH TECHNOLOGY CO., LTD., Weihai, Shandong (CN)

(72) Inventors: Xiaokang LUAN, Weihai, Shandong (CN); Jing ZHU, Weihai, Shandong (CN); Ying WANG, Weihai, Shandong (CN)

(73) Assignee: WEIHAI WEIGAO HEALTH TECHNOLOGY CO., LTD., Weihai, Shandong (CN)

(21) Appl. No.: 19/131,906

(22) PCT Filed: Oct. 12, 2024
(86) PCT No.: PCT/CN2024/124522

§ 371 Date: May 21, 2025

(30) Foreign Application Priority Data
Oct. 12, 2023 (CN) 202322744089.X

(51) Int. Cl. A61M 16/00; F04D 27/00; F04D 29/66; G10K 11/16

(52) U.S. Cl. CPC A61M 16/0003; F04D 27/001; F04D 29/665; F04D 29/667; G10K 11/161; A61M 2205/42

(57) ABSTRACT

A low-noise sound-muffling box for a ventilator, and the ventilator. The low-noise sound-muffling box includes a first housing, a second housing, a housing sealing member, a resistant rib group and a noise reduction mechanism, the first housing and the second housing are spliced to form a whole-box housing, and the whole-box housing is internally provided with an air intake noise reduction chamber and a fan chamber; the resistant rib group divides the air intake noise reduction chamber into at least two air path chambers communicating with each other; the air path chamber on a tail end communicates with the fan chamber; and the noise reduction mechanism is constructed below a fan air inlet.

### CROSS-REFERENCE TO RELATED APPLICATION

The disclosure claims priority and the benefit of Chinese Patent Application No. 202322744089.X, filed with the China National Intellectual Property Administration (CNIPA) on Oct. 12, 2023, and entitled “Low-Noise Sound-muffling box for Ventilator”, which is herein incorporated by reference in its entirety.

### TECHNICAL FIELD

The disclosure relates to the technical field of medical instruments, and in particular to a low-noise sound-muffling box for a ventilator, and the ventilator.

### BACKGROUND

Patients suffering from obstructive sleep apnea and chronic obstructive pulmonary diseases need to use ventilators for adjuvant therapy, and during the therapy, the patients are in a sleep state most of the time and thus require quiet and comfortable surrounding environments. However, the ventilators rely on fans to pressurize air in pipelines to achieve a therapeutic effect on the patients, and the fans inevitably generate noise during operation, thus affecting the sleep of the patients.

The related art mainly achieves the purpose of reducing the noise of the fan by improving the mounting and fixing manner of the fan, mainly reduces aerodynamic noise by designing an internal air flow path in the ventilator, and reduces the noise by setting silencing and sound absorption materials.

Although the above solutions have achieved certain effects, the following problems still exist:

The structure of a fixing device for fixing the fan is relatively complex in general, which directly increases the volume of the ventilator, therefore the usage of the ventilator is restricted by space and position, and the ventilator is inconvenient to move and transport. In order to optimize the air path design, the internal structure of a fan box is complex, which greatly increases the internal air resistance of the ventilator, increases the operation load of the fan and accelerates the equipment loss; in order to stabilize a positive pressure in an air channel, the operation load and operation noise of the fan are increased, and then the loss of the fan is increased; and if the noise reduction and sound absorption materials are configured for a long time, silencer cotton in the silencing material may decompose fragments and release harmful gases, resulting in serious health risks.

### SUMMARY

The objective of the disclosure is to provide a low-noise sound-muffling box for a ventilator, and the ventilator, so as to solve the technical problem in the related art that noise is generated during an operation of ventilator equipment to affect a usage experience of a patient.

The technical solution of the disclosure is:
Some embodiments of the disclosure provide a low-noise sound-muffling box for a ventilator, including a first housing, a second housing, a resistant rib group and a noise reduction mechanism, the first housing and the second housing are hermetically connected by a housing sealing member to form a whole-box housing, and the whole-box housing is internally provided with an air intake noise reduction chamber and a fan chamber;
the first housing and the second housing are distributed in a radial or axial direction of a motor of a fan; and
the resistant rib group is arranged in the air intake noise reduction chamber and divides the air intake noise reduction chamber into at least two air path chambers communicating with each other; an air path chamber on a tail end communicates with the fan chamber; and the fan is suspended in the fan chamber by a fan sleeve, and the noise reduction mechanism is constructed below a fan air inlet.

In some embodiments, the first housing and the second housing are provided with the resistant rib group respectively, and the resistant rib groups in the two housings are symmetrically arranged; and
the resistant rib group includes flow guide rib plates in pairs and compartment rib plates in pairs; and the compartment rib plates in pairs are configured for separating the air path chambers on two sides, the flow guide rib plates in pairs are mounted on the compartment rib plates in pairs in a one-to-one manner and communicate the air path chambers on the two sides, and in response to a plurality of pairs of flow guide rib plates, the plurality of pairs of flow guide rib plates are distributed in a staggered manner.

In some embodiments, the noise reduction mechanism utilizes one or a combination of more of a noise reduction volute, a noise reduction partition plate and a noise reduction resistive material block.

In some embodiments, the noise reduction mechanism is set to be the noise reduction volute, a rotation direction of a flow guide rib of the noise reduction volute is consistent with a rotation direction of an impeller of the fan, and a ratio of an air intake cross-sectional area of the noise reduction volute to a cross-sectional area of the fan air inlet is 90% to 110%; and
a cross section of the noise reduction volute is set to be in a fan blade shape or a vortex line-like shape.

In some embodiments, the noise reduction mechanism is set to be the noise reduction partition plate, micropores are formed in the noise reduction partition plate in an array, and a space is formed in a bottom of the noise reduction partition plate to form a noise reduction chamber.

In some embodiments, the flow guide rib plates in pairs are arranged in parallel or in a flared shape in an air intake direction.

In some embodiments, the flow guide rib plates include first flow guide rib plates and second flow guide rib plates, the at least two air path chambers include a first air path chamber, a second air path chamber and a third air path chamber which communicate with each other in sequence, an air inlet communicating with the first air path chamber is formed in the first housing, the first flow guide rib plates in pairs communicate the first air path chamber with the second air path chamber, and the second flow guide rib plates in pairs communicate the second air path chamber with the third air path chamber.

In some embodiments, the low-noise sound-muffling box further includes an air inlet tube, the air inlet tube is arranged in the first air path chamber and communicates with the air inlet, and the air inlet tube is bent towards a top of the first air path chamber.

In some embodiments, the low-noise sound-muffling box further includes a laminar flow tube, and the laminar flow tube communicates the third air path chamber with the fan chamber.

In some embodiments, a length of the second air path chamber in an air intake direction is L, a length of the first flow guide rib plates is L/4, and a length of the second flow guide rib plates is L/2.

In some embodiments, the low-noise sound-muffling box further includes a differential pressure flow sensor, and probe heads of the differential pressure flow sensor are respectively arranged in the first air path chamber and the fan chamber.

In some embodiments, an air outlet end of the air path chamber on the tail end faces away from a fan air outlet.

In some embodiments, the first housing and the second housing are provided with the resistant rib group respectively, and the resistant rib groups in the two housings are symmetrically arranged; and
the resistant rib group includes a flow guide rib plate and a compartment rib plate; and there are a plurality of flow guide rib plates and a plurality of compartment rib plates, each of the plurality of compartment rib plates is configured for separating the air path chambers on two sides, each of the plurality of flow guide rib plates is mounted on each of the plurality of compartment rib plates in an one-to-one manner, and the plurality of flow guide rib plates are distributed in a staggered manner and form a flow guide channel.

In some embodiments, a first interface of the fan chamber and the air intake noise reduction chamber perpendicularly intersects with a second interface on which the first housing is in butt joint with the second housing.

In some embodiments, the housing sealing member includes a sealing frame and sealing inner slats, an arrangement form of the sealing inner slats is the same as a layout of the resistant rib groups in the first housing and the second housing, and two sides of each of the sealing inner slats are both provided with a groove in sealing butt joint with the flow guide rib plates and the compartment rib plates.

In some embodiments, a support rib group for mounting the fan sleeve is arranged on a peripheral inner wall of the fan chamber; and the support rib group includes at least four fan support ribs, and the at least four fan support ribs are fittingly connected to a ring groove formed in the fan sleeve.

In some embodiments, the fan sleeve includes a sleeve body and a first buffer member, there are a plurality of first buffer members, the plurality of first buffer members are arranged on an outer peripheral wall of the sleeve body at intervals in a circumferential direction, and each of the plurality of first buffer members is detachably connected to an inner wall of the fan chamber.

In some embodiments, the whole-box housing is further internally provided with a buffer chamber, and the buffer chamber communicates with the fan chamber and corresponds to the fan air outlet.

In some embodiments, the low-noise sound-muffling box further includes a second buffer member, and the second buffer member communicates the fan air outlet with the buffer chamber.

In some embodiments, the low-noise sound-muffling box further includes a first pressure sensor, and a probe head of the first pressure sensor is arranged in the buffer chamber.

Some other embodiments of the disclosure provide a ventilator, including a transfer chamber, a water tank and the above low-noise sound-muffling box, the water tank respectively communicates with the low-noise sound-muffling box and the transfer chamber, and the transfer chamber is configured for communicating with a patient end.

In some embodiments, the ventilator further includes a second pressure sensor, a probe head of the second pressure sensor is arranged in the transfer chamber; or the low-noise sound-muffling box further includes an accommodation chamber, the probe head of the second pressure sensor is arranged in the accommodation chamber, and the accommodation chamber communicates with the transfer chamber.

In some embodiments, the ventilator further includes a casing, the water tank is detachably connected to the casing, and a position detection sensor is arranged at a joint of the casing and the water tank for detecting whether the water tank and the casing are connected in place.

Compared with the related art, the disclosure has the following advantages:
(1) In the disclosure, the sound-muffling box is divided into two parts, that is, the first housing and the second housing, the first housing and the second housing are distributed in the radial direction of the fan, a half of the fan chamber and a half of a noise reduction flow channel chamber are both arranged in the first housing and the second housing, and a housing separation line perpendicularly intersects with a chamber separation line, so that the fan is mounted on a side away from an air inlet, an air path is prolonged, a number of times of reflection of noise during propagation is increased, and the noise is thus reduced. Moreover, the noise reduction mechanism is arranged on the bottom of the fan air inlet, and the noise reduction mechanism and the noise reduction flow channel chamber perform resistant composite silencing, thereby improving the noise reduction effect.
(2) A plurality of resistant rib groups are arranged in the flow channel chamber to divide the flow channel chamber into a plurality of small chambers, the small chambers have unequal sizes and are arranged in a high and low staggered manner, and since the plurality of chambers are designed in the staggered manner, the air path is prolonged, and the number of times of reflection of the noise is increased, thereby improving the noise reduction effect.
(3) The noise reduction mechanism utilizes one or a combination of more of the noise reduction volute, the noise reduction partition plate and the noise reduction resistive material block, wherein the cross section of the noise reduction volute is set to be in the fan blade shape or the vortex line-like shape, the rotation direction of the rib plate of the noise reduction volute is consistent with the rotation direction of the impeller of the fan, the ratio of the air intake cross-sectional area of the noise reduction volute is equivalent to the cross-sectional area of the fan air inlet, and flow guide and pressure stabilization are performed on the airflow entering the fan in advance, thereby reducing the turbulence phenomenon at the fan air inlet.
(4) A plurality of support rib groups are arranged on the peripheral side of the fan chamber and the housing sealing member to install the fan sleeve, and the fan is flexibly wrapped and suspended by the fan sleeve, thereby improving the installation stability of the fan, and achieving damping and noise reduction.
(5) A part of the noise generated by the fan is incident on a wall surface of the housing and is absorbed and projected, most of the noise propagates along an air flow path and is finally transmitted out from the air inlet, and the noise at the fan air outlet is reduced by the buffer chamber; and the noise at the fan air inlet is reduced by the laminar flow tube, the second flow guide rib plates and the first flow guide rib plates, thereby realizing effective noise reduction of the whole internal chamber.

### BRIEF DESCRIPTION OF THE DRAWINGS

The disclosure will be further described below in combination with the drawings and embodiments:

FIG. 1 is an exploded view of a low-noise sound-muffling box according to an embodiment of the disclosure;

FIG. 2 is a schematic structural diagram of a first housing according to an embodiment of the disclosure;

FIG. 3 is a schematic structural diagram of a second housing according to an embodiment of the disclosure;

FIG. 4 is a schematic structural diagram of a fan mounted on the second housing according to an embodiment of the disclosure;

FIG. 5 is a stereoscopic schematic diagram of a fan mounted on a housing sealing member according to an embodiment of the disclosure;

FIG. 6 is a schematic diagram of a stereoscopic structure of an overall appearance of a low-noise sound-muffling box according to an embodiment of the disclosure;

FIG. 7 is a sectional view of a noise reduction mechanism of a low-noise sound-muffling box according to Embodiment 1 of the disclosure;

FIG. 8 is a sectional view of a noise reduction mechanism of a low-noise sound-muffling box according to Embodiment 2 of the disclosure;

FIG. 9 is a sectional view of a noise reduction mechanism of a low-noise sound-muffling box according to Embodiment 3 of the disclosure;

FIG. 10 is a discontinuous spiral example diagram of a noise reduction volute of a noise reduction mechanism according to Embodiment 1 of the disclosure;

FIG. 11 is a continuous spiral example diagram of the noise reduction volute of a noise reduction mechanism according to Embodiment 1 of the disclosure;

FIG. 12 is a cambered blade-shaped example diagram of a noise reduction volute of a noise reduction mechanism according to Embodiment 1 of the disclosure;

FIG. 13 is an example diagram of noise reduction of an assembly combination of a noise reduction mechanism of the disclosure;

FIG. 14 is a schematic diagram of an overlooking plane of a noise reduction mechanism of the disclosure;

FIG. 15 is an exploded view of a low-noise sound-muffling box according to another embodiment of the disclosure;

FIG. 16 is a schematic structural diagram of one angle of a first housing and a fan according to another embodiment of the disclosure;

FIG. 17 is a schematic structural diagram of another angle of a first housing and a fan according to another embodiment of the disclosure;

FIG. 18 is a sectional view of a first housing and a fan according to another embodiment of the disclosure;

FIG. 19 is a schematic structural diagram of a first housing and a fan according to yet another embodiment of the disclosure;

FIG. 20 is a schematic structural diagram of a ventilator with a casing omitted of the disclosure;

FIG. 21 is a schematic structural diagram of a ventilator with a casing of the disclosure;

wherein: 1. first housing; 2. second housing; 3. resistant rib group; 4-(a/b/c/d) noise reduction mechanism; 5. housing sealing member; 6. fan sleeve; 7. fan; 8. fan air outflow sealing member;
10. air intake noise reduction chamber; 11. air inlet; 12. air intake extension rib; 20. fan chamber; 21. air outlet; 22. support rib group; 31. flow guide rib plate; 311. first flow guide rib plate; 312. second flow guide rib plate; 32. compartment rib plate;
41. micropores; 42. assembly punch hole; 43. noise reduction chamber;
51. sealing frame; 52. sealing inner slat; 60. ring groove; 61. sleeve body; 62. first buffer member; 71. fan air inlet; 80. air inlet tube; 90. laminar flow tube; 100. air path chamber; 110. buffer chamber; 120. second buffer member; 130. differential pressure flow sensor; 140. first pressure sensor;
101. first finite field; 102. second finite field; 103. third finite field; 104. fourth finite field; 105. first interface; 106. second interface; 107. first air path chamber; 108. second air path chamber; 109. third air path chamber; 520. groove;
1000. low-noise sound-muffling box; 1100. water tank; 1200. transfer chamber; 1300. second pressure sensor; 1400. casing.

### DETAILED DESCRIPTION OF THE EMBODIMENTS

The content of the disclosure will be further described in detail below in combination with specific embodiments:

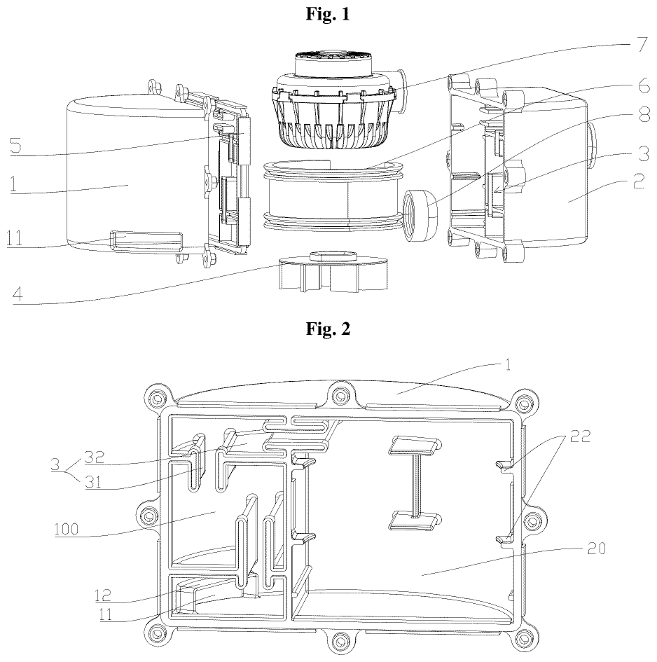

As shown in FIG. 1, a low-noise sound-muffling box for a ventilator includes a first housing 1, a second housing 2, a resistant rib group 3 and a noise reduction mechanism 4, an air inlet 11 is arranged on the first housing 1, and an air outlet 21 is arranged on the second housing 2; and an air intake extension rib 12 is arranged on an inner side of the air inlet 11 (an inner side of the first housing 1).

The first housing 1 and the second housing 2 are divided into two left and right half housings in the radial direction of a fan, with reference to FIG. 6, a butt-joint plane is set to be a second interface 106, and a housing sealing member 5 hermetically connects the first housing 1 and the second housing 2 on the second interface 106 to form a whole-box housing of the sound-muffling box. The whole-box housing is internally provided with two parts, that is, an air intake noise reduction chamber 10 and a fan chamber 20, a separation plane of the fan chamber 20 and the air intake noise reduction chamber 10 is set to be a first interface 105. A fan 7 is suspended in the fan chamber 20 by a fan sleeve 6, and the noise reduction mechanism 4 is constructed below a fan air inlet. The fan 7 is mounted on one side of a box body and is away from the air inlet 11, thereby prolonging an air path and increasing the reflection of noise during propagation, and thus reducing the noise.

The first interface 105 perpendicularly intersects with the second interface 106, which is equivalent to dividing an interior of the whole-box housing into four spatial blocks, and the four spatial blocks are described as a first finite field 101, a second finite field 102, a third finite field 103 and a fourth limit field 104 for the convenience of description.

The first finite field 101 and the third finite field 103 respectively correspond to a half of the air intake noise reduction chamber 10, the second finite field 102 and the fourth limit field 10 respectively correspond to a half of the fan chamber 20, a housing corresponding to the first finite field 101 and the second finite field 102 is the second housing 2, a housing corresponding to the third finite field 103 and the fourth limit field 104 is the first housing 1, and a schematic diagram for mounting the fan 7 in a half of the fan chamber 20 is shown in FIG. 4.

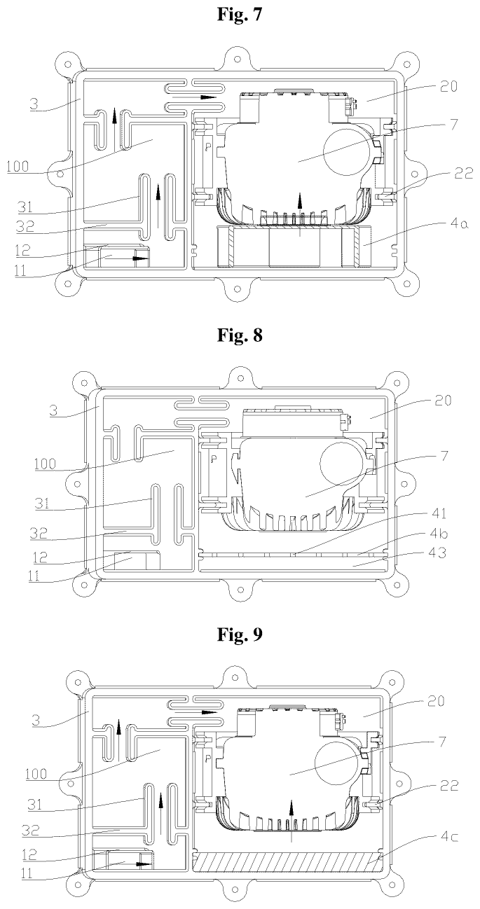

The resistant rib group 3 is arranged in the air intake noise reduction chamber 10, in other words, the first housing 1 and the second housing 2 are provided with the resistant rib group 3 respectively, and the resistant rib groups 3 in the two housings are symmetrically arranged and fit with each other to form an internal air channel of the whole air intake noise reduction chamber 10, that is, the air intake noise reduction chamber 10 is divided into at least two air path chambers 100 communicating with each other, and an air path chamber 100 on a tail end communicates with the fan chamber 20. The air path chambers 100 and an airflow path thereof are shown in FIG. 7.

In an embodiment, the resistant rib group 3 includes flow guide rib plates 31 in pairs and compartment rib plates 32 in pairs; and the compartment rib plates 32 in pairs are configured for separating the air path chambers 100 on two sides, the flow guide rib plates 31 in pairs are mounted on the compartment rib plates 32 in pairs in a one-to-one manner and communicate the air path chambers 100 on the two sides, there are a plurality of pairs of flow guide rib plates, and the plurality of pairs of flow guide rib plates 31 are distributed in a staggered manner and have different lengths, and the separated air path chambers 100 have unequal heights, so that the silencing frequencies of the air path chambers 100 are staggered to each other, thereby improving the noise reduction effect.

In the embodiment, the flow guide rib plates 31 in pairs are arranged in parallel.

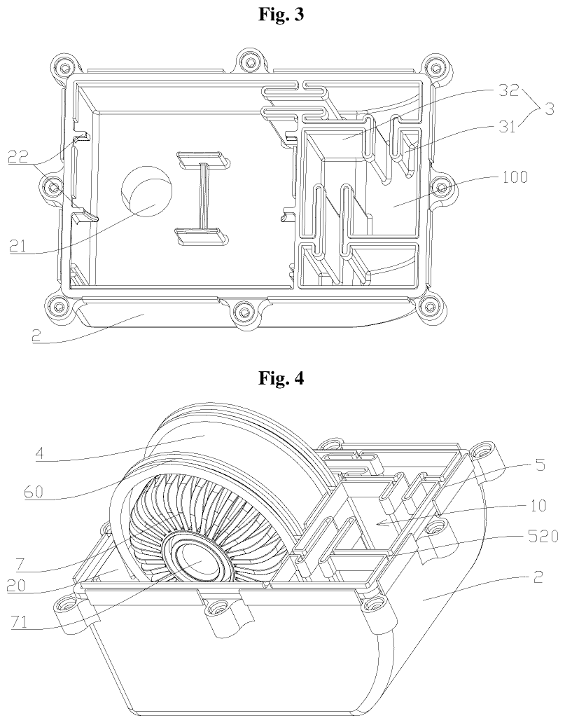

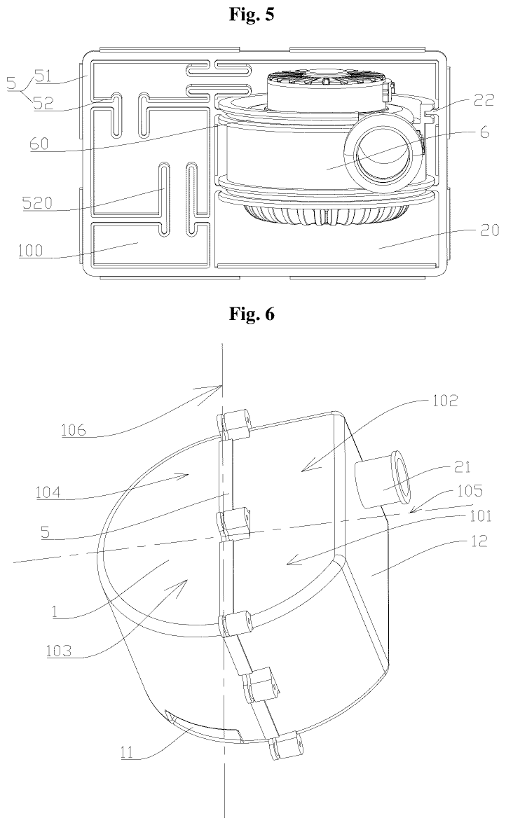

An arrangement of the resistant rib group 3 in the first housing 1 is shown in FIG. 2, an arrangement of the resistant rib group 3 in the second housing is shown in FIG. 3, the housing sealing member 5 forms a butt-joint connection between the resistant rib group 3 in the first housing and the resistant rib group 3 in the second housing 2, so as to divide the air intake noise reduction chamber 10 into three air path chambers 100, and with reference to FIG. 7, there are four large noise reduction chambers in total plus the fan chamber 20; and a structure of the corresponding housing sealing member 5 is shown in FIG. 5.

The housing sealing member 5 includes a sealing frame 51 and sealing inner slats 52, an arrangement form of the sealing inner slats 52 is the same as a layout of the resistant rib groups 3 in the first housing 1 and the second housing 2, and two sides of each of the sealing inner slats 52 are both provided with a groove 520 in sealing butt joint with the flow guide rib plates 31 and the compartment rib plates 32.

The fan 7 is mounted by a fit of lug bosses that are arranged on a peripheral side of the fan in an array and mounting grooves that are formed in an inner wall of the fan sleeve 6 in an array (the lug bosses and the mounting grooves are not shown in the figures), a fan air outflow sealing member 8 is sleeved on an air outlet of the fan, the fan air outflow sealing member 8 is set to be a sleeve, a peripheral side of an end portion of the sleeve abuts against the air outlet 21 of the second housing 2, so as to avoid an airflow flowing out from the fan 7 flowing back to the fan chamber to cause turbulence. Both the fan sleeve 6 and the fan air outflow sealing member 8 are made of a flexible material, such as silica gel, to achieve flexible wrapping and suspension of the fan 7 and to achieve damping and noise reduction.

A support rib group 22 for mounting the fan sleeve 6 is arranged on a peripheral inner wall of the fan chamber 20, the support rib group 22 includes at least four fan support ribs, and with reference to a schematic diagram shown in FIG. 5, in which the fan is mounted on the housing sealing member 5 in a suspended manner, the at least four fan support ribs are fittingly connected to a ring groove 60 formed in the fan sleeve 6.

In the embodiment, eight groups of fan support ribs are provided in total, wherein six groups are arranged on the housing sealing member 5, the first housing 1 and the second housing 2 in a vicinity of the second interface 106, the other two groups are arranged on shell inner walls of the second finite field 102 and the fourth limit field 10 that are away from the second interface 106, the fan support ribs on the inner walls of the housings are set to be H-shaped or N-shaped, and the plurality of groups of fan support ribs have different sizes and different shapes, thereby improving the supporting stability of the fan (sleeve).

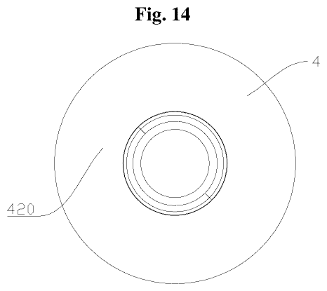

Specific examples in which the noise reduction mechanism 4 is constructed below in the fan are shown in FIGS. 7-13, the noise reduction mechanism 4 includes a support plate body and a noise reduction rib plate body on a bottom, the support plate body and the noise reduction rib plate body are integrally formed, the fan 7 is mounted on an assembly punch hole 42 on the support plate body, and a top view of the noise reduction mechanism is shown in FIG. 14.

Specific embodiments of the noise reduction mechanism 4 are provided below.

### Embodiment 1

The noise reduction mechanism 4-a is set to be a noise reduction volute, and FIG. 7 shows an example diagram of a section on which the noise reduction volute is supported and arranged on the bottom of the fan. A rotation direction of a flow guide rib of the noise reduction volute is consistent with a rotation direction of an impeller of the fan 7, and a ratio of an air intake cross-sectional area of the noise reduction volute to a cross-sectional area of the fan air inlet is 90% to 110%, and an error does not exceed 10%, thereby avoiding the generation of noise caused by relatively large pressure pulsation.

Flow guide and pressure stabilization are performed on the airflow entering the fan in advance, so as to reduce the turbulence phenomenon at the fan air inlet.

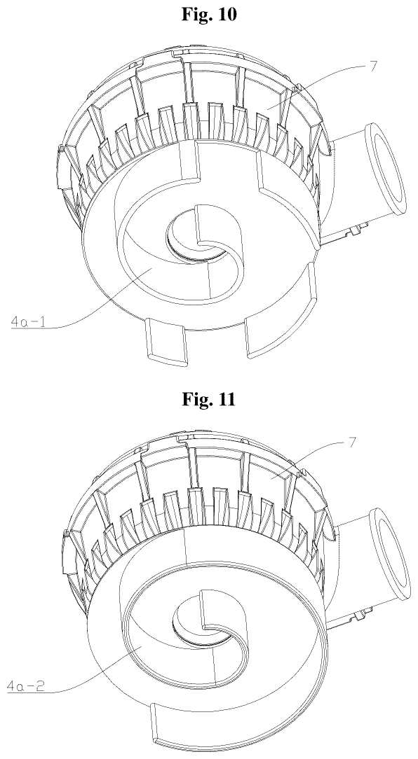

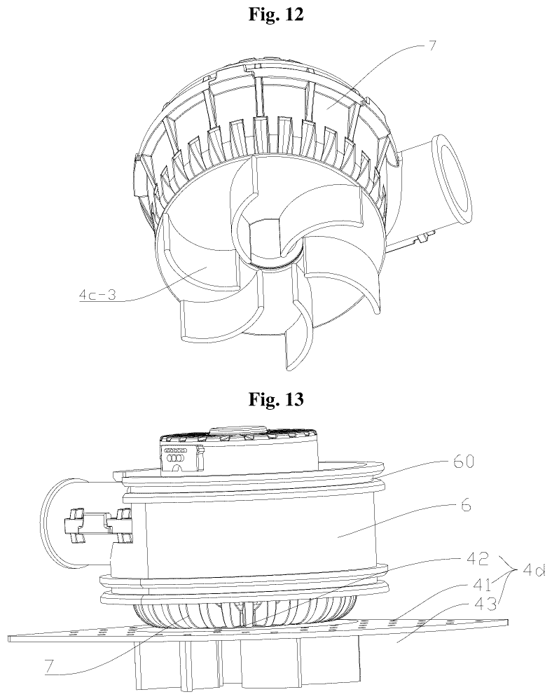

The noise reduction volute may be set to be one of a vortex line-like shape and a fan blade shape, and different forms of the noise reduction volute are shown in FIG. 10, FIG. 11 and FIG. 12. In FIG. 10, the noise reduction volute is set to be in a discontinuous vortex line shape, the noise reduction volute in FIG. 11 is set to be in a continuous vortex line shape, and the noise reduction volute in FIG. 12 is set to be in a fan blade shape.

### Embodiment 2

The noise reduction mechanism 4-b is set to be a noise reduction partition plate, micropores 41 of the same size are formed in an array on the noise reduction partition plate, with reference to FIG. 8, the noise reduction partition plate forms a noise reduction chamber 43 with a space reserved in a bottom of the second housing 2, sound returned from a fan air inlet 71 forms a resonance system with the noise reduction chamber below the micropores 41 through the micropores 41, thereby reducing the noise and eliminating the noise of a specific frequency.

### Embodiment 3

The noise reduction mechanism 4-c is set to be a noise reduction resistive material. With reference to FIG. 9, for the resistive material having a large number of microporous structures therein, sound enters the resistive material to convert sound energy into thermal energy, thereby reducing the energy of the noise.

### Embodiment 4

The noise reduction mechanism 4-d is set to be a combination of the noise reduction volute and the noise reduction partition plate. With reference to FIG. 13, the noise reduction volute is inserted into the noise reduction partition plate, an airflow enters the noise reduction chamber on the bottom from the micropores on the peripheral side of the fan, and enters the fan from the micropores right below the fan, the airflow passes through a noise reduction hole box for noise reduction and turns by 180°, thereby increasing the number of times of collision friction of the sound with the micropores and the plate body, and thus improving the noise reduction effect.

In another embodiment of the disclosure, a low-noise sound-muffling box of another structure is further provided. The main difference from the forgoing embodiments lies in the arrangement manners of the housings and the fan.

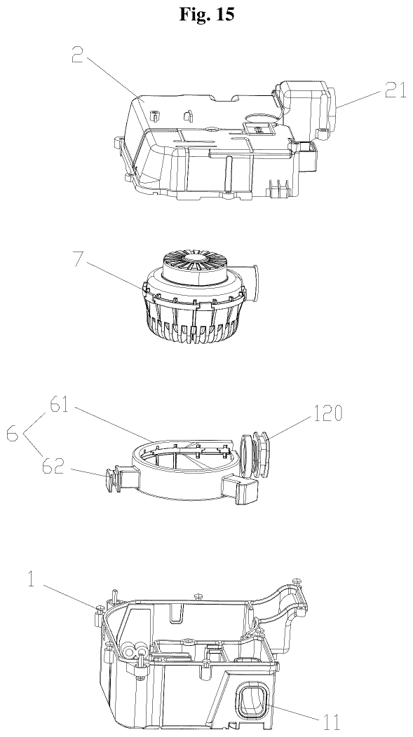

In the embodiment, as shown in FIG. 15, the first housing 1 and the second housing 2 are distributed in an axial direction of a motor of the fan 7. It can be understood that in the foregoing embodiments, the first housing 1 and the second housing 2 are horizontally connected left and right, and in the embodiment, the first housing 1 and the second housing 2 are connected top and down.

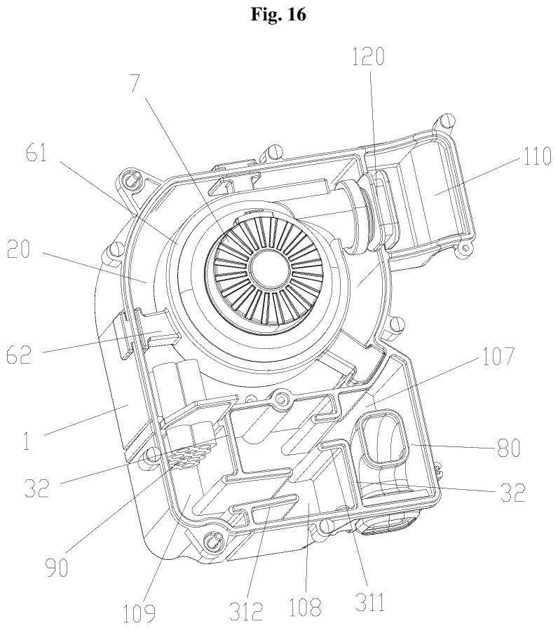

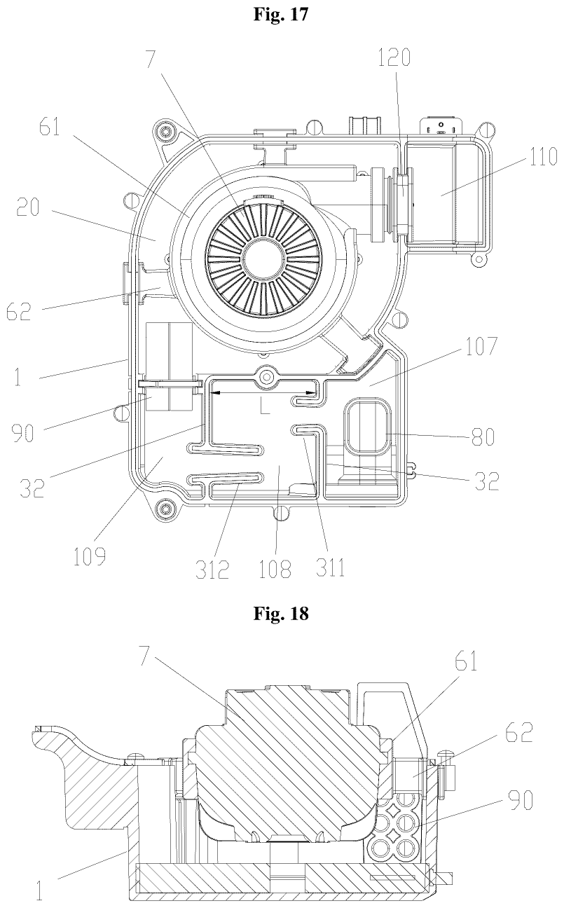

As shown in FIGS. 16-17, the flow guide rib plates 31 include first flow guide rib plates 311 and second flow guide rib plates 312, the air path chambers 100 includes a first air path chamber 107, a second air path chamber 108 and a third air path chamber 109, which communicate with each other in sequence, an air inlet 11 communicating with the first air path chamber 107 is formed in the first housing 11, the first flow guide rib plates 311 in pairs communicate the first air path chamber 107 with the second air path chamber 108, and the second flow guide rib plates 312 in pairs communicate the second air path chamber 108 with the third air path chamber 109.

As shown in FIGS. 16-17, the low-noise sound-muffling box further includes an air inlet tube 80, the air inlet tube 80 is arranged in the first air path chamber 107 and communicates with the air inlet 11, and the air inlet tube 80 is bent towards a top of the first air path chamber 107. It can be understood that the air inlet tube 80 is bent upwards in specific settings.

In the embodiment, air enters from the air inlet tube 80, the air is pressurized by the fan 7 while entering, the pressurized air flows to the top of the first air path chamber 107 under an action of the air inlet tube 80, so that a distance of flowing the fluid towards the side wall of the cavity is prolonged, and a buffer is provided to prevent the generation of regenerative noise caused by the direct collision of the air and the wall.

In the embodiment, during a process of the air touching the top of the first air path chamber 107, a flow rate of the air in a height direction of the low-noise sound-muffling box is slightly different, so that the air is more stable when passing through a first flow guide channel formed by the two first flow guide rib plates 311, the first air path chamber 107 is relatively far away from the fan 7 in an air channel, and the flow rate is relatively slow, but at this time, a positive pressure of the air channel provided by the fan 7 is less affected, and less power consumption is increased; at the same time, due to the relatively small flow rate, the turbulence noise is relatively small; and similarly, the second air path chamber 108 is the same as the first air path chamber 107, after passing through the first flow guide channel, the air first touches a side wall in the vicinity of the second flow guide rib plate 312, and since a length of the second flow guide rib plate 312 is relatively large, the air then touches a side wall in the vicinity of the first flow guide rib plates 311, and then passes through a second flow guide channel formed by two second flow guide rib plates 312 and enters the third air path chamber 109. Although the power consumption may be increased to a certain extent by the above settings, the increased power consumption is relatively small, and due to the relatively small flow rate, the noise is relatively small.

In the embodiment, as shown in FIGS. 16-17, the flow guide rib plates 31 in pairs are in a flared shape in an air intake direction. That is, the first flow guide rib plates 311 in pairs and the second flow guide rib plates 312 in pairs are both set to be the flared shape. In FIG. 17, the air intake direction is a horizontal left direction. By using the flared design, the air resistance is able to be reduced, and the load and power consumption of the fan 7 is able to be reduced.

In the embodiment, as shown in FIGS. 16-18, the low-noise sound-muffling box further includes a laminar flow tube 90, and the laminar flow tube 90 communicates the third air path chamber 109 with the fan chamber 20. By providing the laminar flow tube 90, the air flows into the fan chamber 20 from the third air path chamber 109 in a laminar flow manner, thereby reducing the turbulence degree of the air, and thus reducing the noise.

In the embodiment, as shown in FIG. 17, a length of the second air path chamber 108 in the air intake direction is L, a length of the first flow guide rib plates 311 is L/4, and a length of the second flow guide rib plates 312 is L/2. By means of the above settings, noise sound waves are suddenly expanded on the cross section, resulting in a sudden change in the acoustic resistance, and changing the propagation direction of the sound waves for silencing, so that the silencing frequency band is wider.

In the embodiment, an air outlet end of the air path chamber 100 on the tail end faces away from a fan air outlet. That is, an air outlet end of the third air path chamber 109 is an end facing away from the fan air outlet. The air pre-rotates in the fan chamber 20 and moves in the rotation direction of the fan 7 until being sucked by the fan 7. By means of the above settings, when the air flows into the fan chamber 20 from the third air path chamber 109, the airflow direction is consistent with the rotation direction of the fan 7, thereby reducing the power consumption and noise.

As shown in FIGS. 16-18, the fan sleeve 6 includes a sleeve body 61 and a first buffer member 62. There are a plurality of first buffer members 62, the plurality of first buffer members 62 are arranged on an outer peripheral wall of the sleeve body 61 at intervals in a circumferential direction, and each of the plurality of first buffer members 62 is detachably connected to an inner wall of the fan chamber 20. That is, each of the plurality of first buffer members 62 extends outwards from the outer peripheral wall of the sleeve body 61 and is connected and fixed to the inner wall of the fan chamber 20, so that the fan 7 is suspended in the fan chamber 20. In the above setting manner, an internal space of the fluid is not occupied, the structure is compact, and a buffer role is able to be played on the fan 7, thereby reducing vibration noise generated by the operation of the fan 7.

In the embodiment, as shown in FIGS. 16-17, the whole-box housing is further internally provided with a buffer chamber 110, and the buffer chamber 110 communicates with the fan chamber 20 and corresponds to the fan air outlet. That is, the buffer chamber 110 is located on the most downstream of an internal cavity of the whole low-noise sound-muffling box, and the air outlet 21 is located at the buffer chamber 110. By providing the buffer chamber 110, a flow rate at the air outlet 21 is buffered to a certain extent to reduce the noise at the fan air outlet, so that the air enters the water tank of the ventilator relatively gently.

In the embodiment, as shown in FIGS. 16-17, the low-noise sound-muffling box further includes a second buffer member 120, and the second buffer member 120 communicates the fan air outlet with the buffer chamber 110. The second buffer member 120 is also able to play the buffer role on the fan 7, thereby reducing the vibration noise generated by the operation of the fan 7.

In the embodiment, the first buffer member 62 and the second buffer member 120 are both made of a silica gel material, thereby achieving a better buffer effect.

Since the main noise comes from the operation of the fan 7, a part of the noise generated by the fan 7 is incident on a wall surface of the housing and is absorbed and projected, most of the noise propagates along an air flow path and is finally transmitted out from the air inlet 11, and the noise at the fan air outlet is reduced by the buffer chamber 110; and the noise at the fan air inlet is reduced by the laminar flow tube 90, the second flow guide rib plates 312 and the first flow guide rib plates 311.

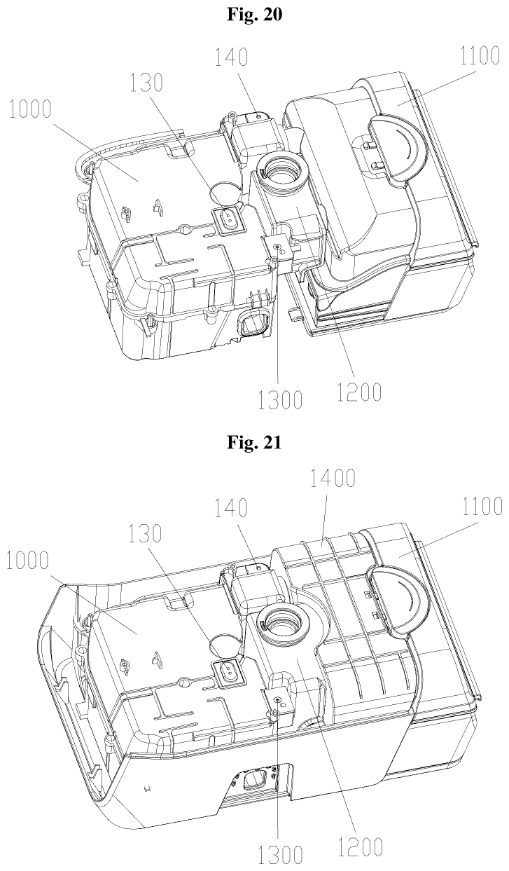

In the embodiment, as shown in FIGS. 20-21, the low-noise sound-muffling box further includes a differential pressure flow sensor 130, and since probe heads of the differential pressure flow sensor 130 need to be located at two places with a certain pressure difference to achieve required recognition precision, and in the disclosure, a pressure difference between the first air path chamber 107 and the fan chamber 20 is the maximum, so that the probe heads of the differential pressure flow sensor 130 are respectively arranged in the first air path chamber 107 and the fan chamber 20, therefore the air resistance of the whole air path and the power consumption of the fan are smaller, and the structure is simple. By providing the differential pressure flow sensor 130, the flow of the air entering a human body from the ventilator is able to be accurately measured, so that accurate flow regulation is able to be performed according to actual requirements.

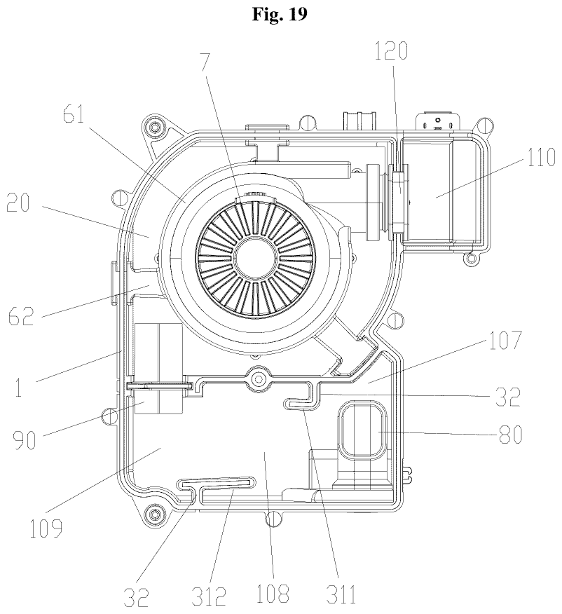

In yet another embodiment of the disclosure, as shown in FIG. 19, the resistant rib group includes a flow guide rib plate 31 and a compartment rib plate 32; and there are a plurality of flow guide rib plates 31 and a plurality of compartment rib plates 32, each of the plurality of compartment rib plates 32 is configured for separating the air path chambers on two sides, each of the plurality of flow guide rib plates 31 is mounted on each of the plurality of compartment rib plates 32 in the one-to-one manner, and the flow guide rib plates 31 are distributed in the staggered manner and form flow guide channels.

That is, in the embodiment, the plurality of flow guide rib plates 31 and the plurality of compartment rib plates 32 are arranged separately. Specifically, the plurality of flow guide rib plates 31 include a first flow guide rib plate 311 and a second flow guide rib plate 312, two compartment rib plates 32 are provided, the first flow guide rib plate 311 and the second flow guide rib plate 312 are respectively connected to the two compartment rib plates 32 and are L-shaped, are respectively located on the upper and lower sides of the air path chamber 100 and are distributed in the staggered manner in the horizontal direction, and a flow guide channel is formed between the first flow guide rib plate 311 and the second flow guide rib plate 312. By means of the above settings, it is possible to avoid the situation that since the structure of the air path chamber 100 is more compact, when the flow guide channel is too narrow and the length of the second air path chamber 108 is too small, the air cannot be buffered in time after being accelerated and thus impacts the inner wall of the chamber at a high speed to generate regenerative noise.

In the embodiment, as shown in FIGS. 20-21, the low-noise sound-muffling box further includes a first pressure sensor 140, and a probe head of the first pressure sensor 140 is arranged in the buffer chamber 110 for monitoring pressure information.

A noise detection result of the low-noise sound-muffling box of the disclosure is shown below.

TABLE 1
Noise detection condition of the low-noise sound-muffling box

| | Hemispherical surface with a radius r of 1.0 m | | | |
|---|---|---|---|---|
| Shape of envelope | Position serial | | | |
| surface | number | x | y | z |
| Position coordinates | 1 | −0.99 | 0.00 | 0.15 |
| of envelope surface | 2 | 0.50 | −0.86 | 0.15 |
| microphone (a | 3 | 0.50 | 0.86 | 0.15 |
| center point of | 4 | −0.45 | 0.77 | 0.45 |
| which is the origin) | 5 | −0.45 | −0.77 | 0.45 |
| | 6 | 0.89 | 0.00 | 0.45 |
| | 7 | 0.33 | 0.57 | 0.75 |
| | 8 | −0.66 | 0.00 | 0.75 |
| | 9 | 0.33 | −0.57 | 0.75 |
| | 10 | 0.00 | 0.00 | 1.00 |

TABLE 2
First noise detection result of the low-noise sound-muffling box
A-weighting sound pressure level and A-weighting sound power level of sample noise band

| | Sound | Sound | Sound | Sound | Sound | Sound |
|---|---|---|---|---|---|---|
| | pressure level | pressure level | pressure level | pressure level | pressure level | pressure level |
| | at point | at point | at point | at point | at point | at point |
| Frequency | location 1 | location 2 | location 3 | location 4 | location 5 | location 6 |
| Hz | dBA(SPL) | dBA(SPL) | dBA(SPL) | dBA(SPL) | dBA(SPL) | dBA(SPL) |
| 100 | −3.22 | −0.49 | −1.30 | −2.57 | −2.33 | −0.26 |
| 125 | 6.26 | 8.83 | 8.02 | 6.65 | 7.01 | 9.01 |
| 160 | 5.93 | 8.43 | 7.67 | 6.18 | 6.75 | 8.71 |
| 200 | 0.29 | 2.48 | 2.00 | 0.71 | 0.92 | 3.00 |
| 250 | 0.37 | 0.73 | 1.35 | 0.50 | 0.11 | 1.83 |
| 315 | 8.15 | 9.02 | 7.71 | 7.99 | 6.09 | 9.35 |
| 400 | 7.84 | 5.88 | 5.18 | 6.61 | 4.77 | 6.64 |
| 500 | 7.03 | 7.23 | 5.39 | 4.90 | 1.35 | 8.00 |
| 630 | 4.44 | 6.09 | 6.20 | 6.90 | 3.53 | 4.17 |
| 800 | 2.77 | 6.24 | 7.52 | 3.91 | 2.73 | 7.37 |
| 1000 | 11.16 | 19.12 | 18.06 | 10.14 | 13.05 | 21.63 |
| 1250 | 10.83 | 13.87 | 11.66 | 9.18 | 9.26 | 14.65 |
| 1600 | 16.62 | 16.64 | 15.21 | 12.24 | 11.52 | 16.19 |
| 2000 | 5.99 | 5.32 | 5.78 | 7.78 | 5.33 | 6.24 |
| 2500 | 18.12 | 12.42 | 16.41 | 15.25 | 18.46 | 16.35 |
| 3150 | 8.79 | 8.11 | 11.06 | 7.73 | 7.99 | 8.52 |
| 4000 | 7.44 | 6.99 | 7.44 | 8.15 | 6.21 | 7.23 |
| 5000 | 8.49 | 7.58 | 9.79 | 8.20 | 8.45 | 7.18 |
| 6300 | 8.93 | 8.05 | 6.60 | 8.17 | 11.54 | 7.19 |
| 8000 | 5.93 | 5.74 | 4.84 | 5.94 | 5.07 | 4.94 |
| 10000 | 4.06 | 4.27 | 4.67 | 4.34 | 3.02 | 3.27 |
| 12500 | 3.29 | 1.36 | 1.10 | 2.16 | 4.91 | 1.17 |
| 16000 | 3.03 | −0.58 | −0.41 | 2.18 | 3.98 | −0.58 |
| 20000 | 1.68 | −3.49 | −4.42 | 0.96 | −2.36 | −4.49 |
| A-weighting | 23.30 | 23.98 | 23.79 | 21.62 | 22.68 | 25.35 |
| sound pressure | | | | | | |
| level | | | | | | |

TABLE 3
Second noise detection result of the low-noise sound-muffling box
A-weighting sound pressure level and A-weighting sound power level of sample noise band

| | Sound | Sound | Sound | Sound | | |
|---|---|---|---|---|---|---|
| | pressure level | pressure level | pressure level | pressure level | Average | |
| | at point | at point | at point | at point | sound | |
| Frequency | location 7 | location 8 | location 9 | location 10 | pressure level | Sound power |
| Hz | dBA(SPL) | dBA(SPL) | dBA(SPL) | dBA(SPL) | dBA(SPL) | dB(PWL) |
| 100 | −1.31 | −2.61 | −0.95 | −1.48 | −1.65 | 6.08 |
| 125 | 7.88 | 6.62 | 8.19 | 7.67 | 7.61 | 15.50 |
| 160 | 7.28 | 6.05 | 7.79 | 6.91 | 7.17 | 15.96 |
| 200 | 1.72 | 0.62 | 1.96 | 1.49 | 1.52 | 9.57 |
| 250 | 1.12 | 0.39 | 0.70 | 0.82 | 0.79 | 8.92 |
| 315 | 7.19 | 7.48 | 7.38 | 6.79 | 7.72 | 15.78 |
| 400 | 5.12 | 6.86 | 4.70 | 5.42 | 5.90 | 14.12 |
| 500 | 5.28 | 6.13 | 3.94 | 5.99 | 5.52 | 13.79 |
| 630 | 7.73 | 5.76 | 3.21 | 8.25 | 5.63 | 14.02 |
| 800 | 6.04 | 1.44 | 4.37 | 5.75 | 4.81 | 13.24 |
| 1000 | 19.22 | 8.79 | 15.32 | 20.04 | 15.65 | 25.51 |
| 1250 | 14.11 | 10.45 | 11.13 | 16.18 | 12.13 | 20.76 |
| 1600 | 14.38 | 12.66 | 13.41 | 18.92 | 14.78 | 23.42 |
| 2000 | 8.08 | 6.34 | 5.69 | 6.78 | 6.33 | 14.44 |
| 2500 | 19.07 | 16.77 | 14.34 | 17.16 | 16.44 | 24.81 |
| 3150 | 9.82 | 8.26 | 8.45 | 8.43 | 8.72 | 16.81 |
| 4000 | 6.88 | 7.79 | 6.57 | 6.09 | 7.08 | 15.14 |
| 5000 | 7.75 | 7.85 | 7.98 | 6.70 | 8.00 | 16.08 |
| 6300 | 7.93 | 8.84 | 6.92 | 7.59 | 8.17 | 16.44 |
| 8000 | 5.05 | 5.58 | 5.40 | 4.95 | 5.34 | 13.37 |
| 10000 | 3.17 | 3.78 | 3.44 | 2.19 | 3.62 | 11.61 |
| 12500 | 1.73 | 2.51 | 2.81 | 1.35 | 2.24 | 10.43 |
| 16000 | 0.48 | 1.31 | 0.92 | 0.34 | 1.07 | 9.28 |
| 20000 | −0.10 | −3.36 | −2.85 | −1.70 | −2.01 | 5.86 |
| A-weighting sound | 24.72 | 21.95 | 22.16 | 25.36 | 23.49 | / |
| pressure level | | | | | | |

It is calculated from the above tables that the A-weighting average sound pressure level Lp of the low-noise sound-muffling box of the disclosure is 23.49 dBA (SPL), and the A-weighting sound power level Lw is 31.7 dB (PWL), therefore the low-noise sound-muffling box of the disclosure has a good noise reduction and silencing effect.

As shown in FIGS. 20-21, the disclosure further provides a ventilator, including a transfer chamber 1200, a water tank 1100 and the above low-noise sound-muffling box 1000, the water tank 1100 respectively communicates with the low-noise sound-muffling box 1000 and the transfer chamber 1200, and the transfer chamber 1200 is configured for communicating with a patient end by a heating respiration pipeline and a patient interface.

In the embodiment, as shown in FIGS. 20-21, the ventilator further includes a second pressure sensor 1300, a probe head of the second pressure sensor 1300 is arranged in an accommodation chamber, the accommodation chamber is arranged on an outer side wall of the low-noise sound-muffling box, and the accommodation chamber communicates with the transfer chamber 1200, so that the second pressure sensor 1300 is configured for detecting the pressure of the transfer chamber 1200. In another embodiment, the probe head of the second pressure sensor 1300 may also be directly arranged in the transfer chamber 1200, which is able to be selected according to actual requirements. By means of the above settings, the second pressure sensor 1300 is close to the patient end, so that pressure information can be accurately monitored. The probe head of the first pressure sensor 140 is arranged in the buffer chamber 110 so as not to be affected by humidified air, thus the probe head is unlikely to be damaged, and a single fault caused by the damage to one of the pressure sensors is prevented.

In the embodiment, as shown in FIGS. 20-21, the ventilator further includes a casing 1400, the water tank 1100 is detachably connected to the casing 1400, and a position detection sensor is arranged at a joint of the casing 1400 and the water tank 1100 for detecting whether the water tank 1100 and the casing 1400 are connected in place.

In the embodiment, the position detection sensor is a microswitch, and the installation position of the position detection sensor is not limited, as long as the position detection sensor is arranged at the joint of the casing 1400 and the water tank 1100, for example, corresponding inner side walls of the casing 1400 and the water tank 1100, and further, the position detection sensor is located at a position close to a lower end on the inner side wall to avoid an air inlet and an air outlet, so as to avoid the damage of the heated and humidified air to the sensor. The ventilator further includes an indicator light and a PCBA board, and the microswitch is electrically connected to the indicator light and the PCBA board, respectively. After the water tank 1100 and the casing 1400 are connected in place, the indicator light is turned on and is controlled by a circuit, and the indicator light is turned off after a certain time, thereby avoiding light pollution; if the water tank 1100 and the casing 1400 are not connected in place, the indicator light is not turned on, and the user needs to check the ventilator to solve the problem.

The above embodiments are only configured for illustrating the technical concepts and features of the disclosure, and are intended to enable those skilled in the art to understand the content of the disclosure and implement the same accordingly, but are not intended to limit the protection scope of the disclosure. It will be apparent to those skilled in the art that the disclosure is not limited to the details of the above specific embodiments, and that the disclosure may be implemented in other specific forms without departing from the spirit or essential characteristics of the disclosure, therefore the embodiments should be regarded as exemplary and nonrestrictive from any perspective, and the scope of the disclosure is defined by the appended claims rather than the foregoing description, therefore it is intended that all changes falling within the meaning and scope of equivalents of the claims are to be embraced within the disclosure.

1. A low-noise sound-muffling box for a ventilator, comprising a first housing, a second housing, a resistant rib group and a noise reduction mechanism, wherein the first housing and the second housing are hermetically connected by a housing sealing member to form a whole-box housing, and the whole-box housing is internally provided with an air intake noise reduction chamber and a fan chamber;
the first housing and the second housing are distributed in a radial or axial direction of a motor of a fan; and
the resistant rib group is arranged in the air intake noise reduction chamber and divides the air intake noise reduction chamber into at least two air path chambers communicating with each other; an air path chamber on a tail end communicates with the fan chamber; and the fan is suspended in the fan chamber by a fan sleeve, and the noise reduction mechanism is constructed below a fan air inlet.

2. The low-noise sound-muffling box for the ventilator according to claim 1, wherein the first housing and the second housing are provided with the resistant rib group respectively, and the resistant rib groups in the two housings are symmetrically arranged; and
the resistant rib group comprises flow guide rib plates in pairs and compartment rib plates in pairs; and the compartment rib plates in pairs are configured for separating the air path chambers on two sides, the flow guide rib plates in pairs are mounted on the compartment rib plates in pairs in a one-to-one manner and communicate the air path chambers on the two sides, and in response to a plurality of pairs of flow guide rib plates, the plurality of pairs of flow guide rib plates are distributed in a staggered manner.

3. The low-noise sound-muffling box for the ventilator according to claim 2, wherein the noise reduction mechanism utilizes one or a combination of more of a noise reduction volute, a noise reduction partition plate, and a noise reduction resistive material block.

4. The low-noise sound-muffling box for the ventilator according to claim 3, wherein the noise reduction mechanism is set to be the noise reduction volute, a rotation direction of a flow guide rib of the noise reduction volute is consistent with a rotation direction of an impeller of the fan, and a ratio of an air intake cross-sectional area of the noise reduction volute to a cross-sectional area of the fan air inlet is 90% to 110%; and
a cross section of the noise reduction volute is set to be in a fan blade shape or a vortex line-like shape.

5. The low-noise sound-muffling box for the ventilator according to claim 3, wherein the noise reduction mechanism is set to be the noise reduction partition plate, micropores are formed in the noise reduction partition plate in an array, and a space is formed in a bottom of the noise reduction partition plate to form a noise reduction chamber.

6. The low-noise sound-muffling box for the ventilator according to claim 2, wherein the flow guide rib plates in pairs are arranged in parallel or in a flared shape in an air intake direction.

7. The low-noise sound-muffling box for the ventilator according to claim 2, wherein the flow guide rib plates comprise first flow guide rib plates and second flow guide rib plates, the at least two air path chambers comprise a first air path chamber, a second air path chamber and a third air path chamber which communicate with each other in sequence, an air inlet communicating with the first air path chamber is formed in the first housing, the first flow guide rib plates in pairs communicate the first air path chamber with the second air path chamber, and the second flow guide rib plates in pairs communicate the second air path chamber with the third air path chamber.

8. The low-noise sound-muffling box for the ventilator according to claim 7, wherein the low-noise sound-muffling box further comprises:
an air inlet tube, wherein the air inlet tube is arranged in the first air path chamber and communicates with the air inlet, and the air inlet tube is bent towards a top of the first air path chamber; and/or
a laminar flow tube, wherein the laminar flow tube communicates the third air path chamber with the fan chamber.

9. (canceled)

10. The low-noise sound-muffling box for the ventilator according to claim 7, wherein a length of the second air path chamber in an air intake direction is L, a length of the first flow guide rib plates is L/4, and a length of the second flow guide rib plates is L/2.

11. The low-noise sound-muffling box for the ventilator according to claim 7, wherein the low-noise sound-muffling box further comprises a differential pressure flow sensor, and probe heads of the differential pressure flow sensor are respectively arranged in the first air path chamber and the fan chamber.

12. The low-noise sound-muffling box for the ventilator according to claim 1, wherein an air outlet end of the air path chamber on the tail end faces away from a fan air outlet.

13. The low-noise sound-muffling box for the ventilator according to claim 1, wherein the first housing and the second housing are provided with the resistant rib group respectively, and the resistant rib groups in the two housings are symmetrically arranged; and
the resistant rib group comprises a flow guide rib plate and a compartment rib plate; and there are a plurality of flow guide rib plates and a plurality of compartment rib plates, each of the plurality of compartment rib plates is configured for separating the air path chambers on two sides, each of the plurality of flow guide rib plates is mounted on each of the plurality of compartment rib plates in an one-to-one manner, and the plurality of flow guide rib plates are distributed in a staggered manner and form a flow guide channel.

14. The low-noise sound-muffling box for the ventilator according to claim 1, wherein a first interface of the fan chamber and the air intake noise reduction chamber perpendicularly intersects with a second interface on which the first housing is in butt joint with the second housing.

15. The low-noise sound-muffling box for the ventilator according to claim 2, wherein the housing sealing member comprises a sealing frame and sealing inner slats, an arrangement form of the sealing inner slats is the same as a layout of the resistant rib groups in the first housing and the second housing, and two sides of each of the sealing inner slats are both provided with a groove in sealing butt joint with the flow guide rib plates and the compartment rib plates.

16. The low-noise sound-muffling box for the ventilator according to claim 1, wherein a support rib group for mounting the fan sleeve is arranged on a peripheral inner wall of the fan chamber; and the support rib group comprises at least four fan support ribs, and the at least four fan support ribs are fittingly connected to a ring groove formed in the fan sleeve.

17. The low-noise sound-muffling box for the ventilator according to claim 1, wherein the fan sleeve comprises a sleeve body and a first buffer member, there are a plurality of first buffer members, the plurality of first buffer members are arranged on an outer peripheral wall of the sleeve body at intervals in a circumferential direction, and each of the plurality of first buffer members is detachably connected to an inner wall of the fan chamber.

18. The low-noise sound-muffling box for the ventilator according to claim 1, wherein the whole-box housing is further internally provided with a buffer chamber, and the buffer chamber communicates with the fan chamber and corresponds to the fan air outlet.

19. The low-noise sound-muffling box for the ventilator according to claim 18, wherein the low-noise sound-muffling box further comprises:
a second buffer member, wherein the second buffer member communicates the fan air outlet with the buffer chamber; and/or
a first pressure sensor, wherein a probe head of the first pressure sensor is arranged in the buffer chamber.

20. (canceled)

21. A ventilator, comprising a transfer chamber, a water tank and the low-noise sound-muffling box according to claim 1, wherein the water tank respectively communicates with the low-noise sound-muffling box and the transfer chamber, and the transfer chamber is configured for communicating with a patient end.

22. The ventilator according to claim 21, wherein the ventilator further comprises:
a second pressure sensor, wherein a probe head of the second pressure sensor is arranged in the transfer chamber; or
the low-noise sound-muffling box further comprises an accommodation chamber, the probe head of the second pressure sensor is arranged in the accommodation chamber, and the accommodation chamber communicates with the transfer chamber;
a casing, wherein the water tank is detachably connected to the casing, and a position detection sensor is arranged at a joint of the casing and the water tank for detecting whether the water tank and the casing are connected in place.

23. (canceled)
